(19) United States
(12) Patent Application Publication
(10) Pub. No.: US 2012/0017915 A1
(43) Pub. Date: Jan. 26, 2012

(54) RESPIRATORY DEVICE COMPRISING A DOUBLE LUMEN ENDOTRACHEAL TUBE

(75) Inventor: Hajo Reissmann, Hamburg (DE)

(73) Assignee: Hajo Reissmann

(21) Appl. No.: 13/236,146

(22) Filed: Sep. 19, 2011

Related U.S. Application Data
(63) Continuation-in-part of application No. 10/555,142, filed on Jun. 4, 2008, now Pat. No. 8,025,059.

(30) Foreign Application Priority Data
Apr. 30, 2003 (DE) 10319384.7

(51) Int. Cl. A61M 16/04; A61M 16/06; A61M 16/01

(52) U.S. Cl. 128/207.14; 128/203.12; 128/205.17; 128/205.25

(57) ABSTRACT

Device for ventilation with a double lumen endotracheal tube featuring a connecting piece at the end distal to the patient into which the two lumina of the endotracheal tube are extended, separated within the connecting piece by an axial partition wall, a connector with two nozzles leading to a joint connecting section and separated from each other by another partition wall, the connector being attachable to the connecting piece in a way that brings the partition wall and the other partition wall into a sealing contact, a ventilator and at least one tube connected to a nozzle of the connector at one end and to the ventilator at the other.

This application is a continuation-in-part of U.S. application Ser. No. 10/555,142, filed Jun. 4, 2008, which is a National Stage of International Application PCT/EP2004/003935, filed Apr. 15, 2004, which claims the benefit of German Patent Application No. 10319384.7, filed Apr. 30, 2003; the contents of which are herein incorporated by reference in their entirety.

The invention relates to a device for ventilation via a double lumen endotracheal tube.

The majority of patients being ventilated for intensive care or anesthesia has their airway secured by an endotracheal tube. The latter usually is made from rubber or plastic. It is inserted either through the natural upper airways, i.e. mouth or nose, pharynx, and larynx, or by surgical access to the trachea, i.e. tracheostomy. Generally, the endotracheal tube is equipped with an inflatable cuff around its tracheal end, which seals the trachea and thus allows positive pressure ventilation and protects the airway from contamination by foreign matter. The end of the tube distal to the patient is either coupled to a ventilator via a tubing system or open to ambient air via a check valve at its outlet.

Common single lumen endotracheal tubes are equipped with a standardized joining piece, a nearly cylindrical cone approximately 10 mm long with an outer diameter of 15 mm. The inspiratory and expiratory tubes are joined in a Y-connector, which offers a corresponding female cone into which the joining piece of the endotracheal tube can be inserted, forming an airtight connection.

With double lumen endotracheal tubes, as described in WO 02/089885 A, usually each lumen is connected via its own standardized joining piece and a straight connector with corresponding female cone to the inspiratory and the expiratory tube, respectively.

This type of connection, however, is impractical and poses safety hazards: Two connectors double the risk of disconnection. Furthermore, two pairs of joining pieces and connectors of identical dimensions may lead to confusion of the two lumina. Color coding or similar marks offer merely limited protection in this respect. A connection with a ventilator using a conventional Y-connector is not readily feasible.

In case of a malfunction jeopardizing regular delivery of gas to the patient, conventional ventilators in connection with single lumen endotracheal tubes open a safety valve in their inspiratory limb, at least allowing the patient to breathe ambient air, if he is physically able. The expiratory valve usually allows expiration in this case of malfunction, and both valves cooperate to secure the necessary unidirectional flow of air. With a double lumen endotracheal tube, one lumen each is available for inspiration and expiration, respectively, also in this case of malfunction. The patient would be confronted with relatively high flow resistances, especially if the double lumen endotracheal tube would feature an asymmetrical design with a narrower inspiratory lumen, which from the standpoint of regular ventilation is the preferred design.

Accordingly the invention is based on the task to create a device allowing ventilation via a safe and easy to operate connection between a double lumen endotracheal tube and a ventilator.

This problem is solved by a device for ventilation having the features of the various embodiments described herein.

A device for ventilation constructed according to the inventions can be comprised of

1. a double lumen endotracheal tube with a joining piece at the end distal to the patient, into which the two lumina of the endotracheal tube are inserted, being separated by an axial partition wall within the joining piece,

2. a connector with two openings for ventilator tubing leading to an opening accepting the above mentioned joining piece, another axial partition wall within the connector forming a continuous sealing partition together with the partition wall of the joining piece when the latter is inserted into the connector,

3. a ventilator, and

4. at least one tube leading from the ventilator to one of the openings of the connector.

A device according to the invention allows to separately connect the two lumina of the endotracheal tube to the two limbs of a ventilator tubing system or to an inspiratory tube and an expiratory valve leading into the atmosphere via a suitable connector. For this purpose the two lumina are continued into the joining piece and separated therein by an axial partition wall, likewise the tubing system limbs are continued towards the opening within the connector accepting the joining piece and separated by another axial partition wall. Furthermore, when the joining piece is introduced into the connector the partition wall will come into sealing contact with the other partition wall, securing a separation of inspiratory and expiratory airways throughout ventilator tubing and endotracheal tube. With only one connection between endotracheal tube and ventilator handling is easier and the risk of an inadvertent disconnection is reduced. In principle the double lumen endotracheal tube can be connected to ventilator through a conventional Y-connector, e.g. in case a ventilator with a connector described in this invention is not available.

According to one embodiment the joining piece has a circular external circumference and the opening accepting it a corresponding circular internal circumference, like in the standardized Y-connectors. According to another embodiment the joining piece and the accepting opening are conical or cylindrical.

According to one embodiment the connector is Y-shaped, as conventional connectors, or T-shaped. In principle it is possible to construct the connector from a conventional Y-connector and an adapter introduced into its cone which incorporates the other partition wall and a cone of its own accepting the joining piece.

The seal between the partition wall and the other partition wall can be obtained in several ways. For this purpose according to one embodiment the walls meet bluntly or overlap partially. According to another embodiment soft elastic seals become effective between the walls.

The partition walls can have various cross sections. According to one embodiment the partition walls are shaped like plates or tubes. A conical or cylindrical joining piece and a conical or cylindrical connector opening are partitioned into lumina with circle segment cross sections by plate-shaped partition walls. Tube-shaped partition walls are preferably located in a concentric position within the joining piece and the connector opening.

Various components of the device can advantageously have standardized dimensions, such as a standardized internal diameter and/or a standardized external diameter, and such dimensions may particularly correspond to dimensions of like parts in the industry that allow for common connection of the present device to existing parts, such as existing ventilator connections and/or existing patient side breathing components—e.g., endotracheal tubes and breathing masks (or face masks). As used herein, a standardized dimension may mean a dimension that is industry recognized as being used so as to allow connections of parts that are recognized as being used together but may be made by different manufacturers. According to one embodiment the joining piece has an external diameter of approximately 15 mm, the connector opening having a corresponding internal diameter. According to another embodiment the joining piece has an internal diameter of approximately 22 mm and the connector opening an external diameter of approximately 22 mm. These embodiments allow the double lumen endotracheal tube to be used with conventional connectors at the ventilator tubing and the connector to be used with conventional single lumen endotracheal tubes or ventilation masks. They make use of the fact that there are two standards for the connection of tubing, Y-connectors, etc. to endotracheal tubes, ventilation masks etc.:

15 mm, female at the side of the ventilator (breathing bag, . . . ), male towards the patient—usually but not exclusively used for endotracheal tubes, and

22 mm, male at the side of the ventilator (breathing bag, . . . ), female towards the patient—usually but not exclusively used for ventilation masks.

According to one embodiment a tube-shaped partition wall in the joining piece has an external diameter of approximately 15 mm, and the other partition wall in the connector opening is tube-shaped with an internal diameter of approximately 15 mm, the entire joining piece has an internal diameter of approximately 22 mm and the connector opening an external diameter of approximately 22 mm. In this way the patient side (at the double lumen endotracheal tube) has both a 15 mm male and a 22 mm female part; towards the ventilator (breathing bag, . . . ), i.e. there is the connector with a 15 mm female as well as a 22 mm male part. The latter in this respect resembles conventional connectors, several of which have an opening complying with both standards (i.e. forming a nozzle of 15 mm internal diameter and 22 mm external diameter) in order to flexibly function together with a variety of endotracheal tubes, ventilation masks etc. According to the invention both standards can be applied at the same time for separately connecting the two lumina of the endotracheal tube to the two limbs of the tubing via the lumen within the tube-shaped partition walls and the ring-shaped lumen around the partition walls, respectively. Although not necessary, using the interior lumen with its full cross section for expiration is advantageous, since it allows unimpeded passage of a suction catheter. In this case inspiration would take place through the ring-shaped exterior lumen. An additional advantage of such solution is the rotational symmetry allowing connection in arbitrary rotational positions.

Such connector can be used with conventional single lumen endotracheal tubes, if a valve of suitable construction makes sure that the circular lumen is blocked and a shortcut between the two lumina is opened. Furthermore the joining piece can be used with a conventional Y-connector. If both lumina of the endotracheal tube are to be used, a shortcut between the two lumina has to be provided, e.g. by a valve. This is, however, not urgently necessary, because, in the preferred embodiment, the central lumen of the joining piece is continued into the wider one of two unequally wide endotracheal tube lumina.

According to one embodiment the joining piece has at least one groove and the connector at least one complementary projection or vice versa, which articulate when the joining piece is inserted into the connector. By their arrangement they allow a connection only in a defined rotational orientation, thus ensuring a correct connection of the endotracheal tube lumina with the inspiratory and expiratory limb of the ventilator tubing, respectively. In principle the correct connection can also be secured by plate-shaped partition walls overlapping each other and being situated eccentrically within joining piece and connector. Furthermore this is secured by partition walls forming concentric circular tubes; these would allow arbitrary rotational positions.

According to one embodiment the connector and/or the joining piece contain a shutter bridging the two sides of the partition wall or the other partition wall. This shutter can be opened in case of ventilator malfunction in order to allow spontaneous breathing via both lumina of the endotracheal tube. This makes sure that a spontaneously breathing patient is not confronted with unduly high resistances, especially if the double lumen endotracheal tube is asymmetrical by design.

In addition, connecting the two tubing limbs by opening the shutter is advantageous for the following reasons: In case of a leakage somewhere between ventilator and patient, a ventilator in a pressure-regulated mode of ventilation tries to restitute the set pressure by increasing gas delivery up to the technically feasible maximum. In case of large leaks, e.g. a disconnection, this usually will not be successful. In case of a disconnection within the expiratory limb during ventilation through a double lumen endotracheal tube, the compensatory delivery of gas is directed through the endotracheal tube, i.e. through the trachea. Depending on the resistance of the expiratory limb of the endotracheal tube and the parts between endotracheal tube and the site of disconnection unwanted high pressures might ensue. In this case of malfunction a shortcut between the tubing limbs is useful.

According to one embodiment the closing element of the shutter has its seat in the partition wall and/or the other partition wall. By moving the closing element away from its seat the partitioning wall is opened. The closing element can e.g. be a valve cone or a flap.

According to one embodiment the shutter can be operated manually from the outside by a suitable actuator. The manual operation can be performed by the medical personnel. According to one embodiment the shutter has an operating connection with a control component of the ventilator. Operation of the shutter by the ventilator can be implemented with a high degree of reliability. According to one embodiment the shutter has an operating connection with a pressure sensor in the expiratory or inspiratory limb, e.g. within the ventilator tubing. Under normal conditions the pressure at these sites is more or less positive. Negative pressures mean a malfunction. This solution avoids the unreliable operation by band as well as modifications to the ventilator. In principle the various alternatives for operating the shutter can be freely combined.

According to one embodiment the shutter has a mark which is visible from the outside and shows at all times, whether the two lumina of the double lumen endotracheal tube are used separately in their respective phases, i.e. as intended, or simultaneously. According to one embodiment the shutter has a sensor for the position of the closing element connected to the control component of the ventilator. This way the status of the shutter can automatically be taken into account during regulation of ventilation and/or display of operating data and/or alarm conditions.

According to one embodiment the connector features joints for the tubing which can be swiveled around at lest one axis. This allows the tubes to approach the connector from a wide range of angles. Rotating or swiveling joints with one or more axes or ball joints are conceivable.

According to one embodiment the connector is equipped with a closable opening which, when opened, allows the insertion of a suction catheter into the expiratory lumen of the endotracheal tube. Due to the uninterrupted connection the suction catheter can be inserted into the expiratory lumen of the double lumen endotracheal tube during ongoing ventilation. According to a simple embodiment the device is equipped with a fastener which can seal the opening. In another embodiment the opening is spanned by a perforated elastic membrane, through which the suction catheter can be introduced without producing a leak, so the desired positive pressure in the lungs can be maintained. The perforated elastic membrane is able to adapt to catheters of various diameters.

According to another embodiment, a double lumen connector device can provide cross-connectivity between a ventilation device and a single lumen or double lumen patient side breathing component. Exemplary ventilation devices can include ventilators, anesthesia machines, resuscitation bags, and the like. In such embodiments, the double lumen connector device can comprise two pieces that slidably engage to form the single device and that slidably disengage to allow separate use. In one embodiment, the joining piece is separate from the endotracheal tube and is a component piece of the device. Thus, the device comprises a joining piece that can include first and second tubes that can be aligned together so as to define a machine end that can be configured for attachment to a ventilator machine (or ventilator connecting piece) and an opposing patient end that is configured for attachment to a double lumen endotracheal tube. For example, the first and second tubes may be axially oriented and spaced apart inner and outer tubes, respectively. The first tube (e.g., the inner tube) can have a first standardized external diameter (e.g., about 15 mm, as described above), and the second tube (e.g., the outer tube) can have a first standardized internal diameter (e.g., about 22 mm, as described above). When the first and second tubes are axially oriented, it can be preferable for the outer tube at the machine end to extend beyond the inner tube at the machine end. The device further can comprise a connector piece that has a patient end that slidably engages the machine end of the joining piece and that has a machine end having separate inspiratory and expiratory tube nozzles extending therefrom and intersecting within the connector piece. The connector piece also includes first and second chambers formed therein by the intersecting nozzles, and the chambers remain separated from each other by a partition wall that extends from within the connector piece to the patient end of the connector piece. The patient end of the connector piece can have a second standardized internal diameter (e.g., about 15 mm, as described above) that preferably corresponds to the external diameter of the first tube of the joining piece. The patient end of the connector piece also can have a second standardized external diameter (e.g., about 22 mm, as described above) that preferably corresponds to the internal diameter of the second tube of the joining piece.

The above construction with the incorporation of standardized sizing provides for cross-connectivity in that the two separate pieces of the connector can be separately used with conventional, single lumen breathing apparatuses. For example, the joining piece of the device can be used separate from the connector piece to allow cross connection of a double lumen endotracheal tube with a standard, single lumen ventilator connection. Similarly, the connector piece can be used separate from the joining piece to allow cross connection with a standard, single lumen patient side breathing component, such as a single lumen endotracheal tube or a breathing mask. Specifically, a section of the partition wall within the connector piece can be configured for translocation from a first position wherein the chambers remain separated within the connector piece to a second position (e.g., within the connector piece or outside of the connector piece) allowing fluid communication between the chambers within the connector piece. When fluid communication between the chambers is facilitated, the double lumen connector piece thus can essentially function as a single lumen connector piece.

In light of the foregoing, in certain embodiments, there is provided a double lumen endotracheal tube that cross connects with a patient end of a standard, single lumen ventilator connection piece. In particular, the double lumen endotracheal tube can be as further described herein or as known in the art and can comprise a first lumen, a separate, second lumen, a first end that is proximal to a patient, and a second end that is distal to the patient. The double lumen endotracheal tube further can comprise a joining piece that can include first and second tubes, a machine end that is configured for attachment to a standard, single lumen ventilator, and a patient end that is configured for attachment to the end of the double lumen endotracheal tube that is distal to the patient. The structure of the joining piece can otherwise be as described above. Preferably, when the first and second tubes are configures as axially oriented and spaced apart inner and outer tubes, the outer tube at the machine end preferably extends beyond the inner tube at the machine end a sufficient distance such that, when the joining piece is connected to the patient end of the standard, single lumen ventilator connection piece, the inner tube of the joining piece remains spaced apart therefrom. The joining piece may be separable from the double lumen endotracheal tube (i.e., discrete components) or may be integral therewith (i.e., non-separable).

Further in light of the foregoing, in certain embodiments, there is provided a ventilator connector piece configured for cross connectivity of a ventilation device with a single lumen endotracheal tube or a double lumen endotracheal tube. The connector piece can be configured as already described above, particularly including a partition wall wherein a section thereof is configured for translocation from a first position wherein the chambers remain separated within the connector piece to a second position allowing fluid communication between the chambers within the connector piece.

Subsequently the invention is illustrated using the accompanying drawings of embodiment examples. The drawings show:

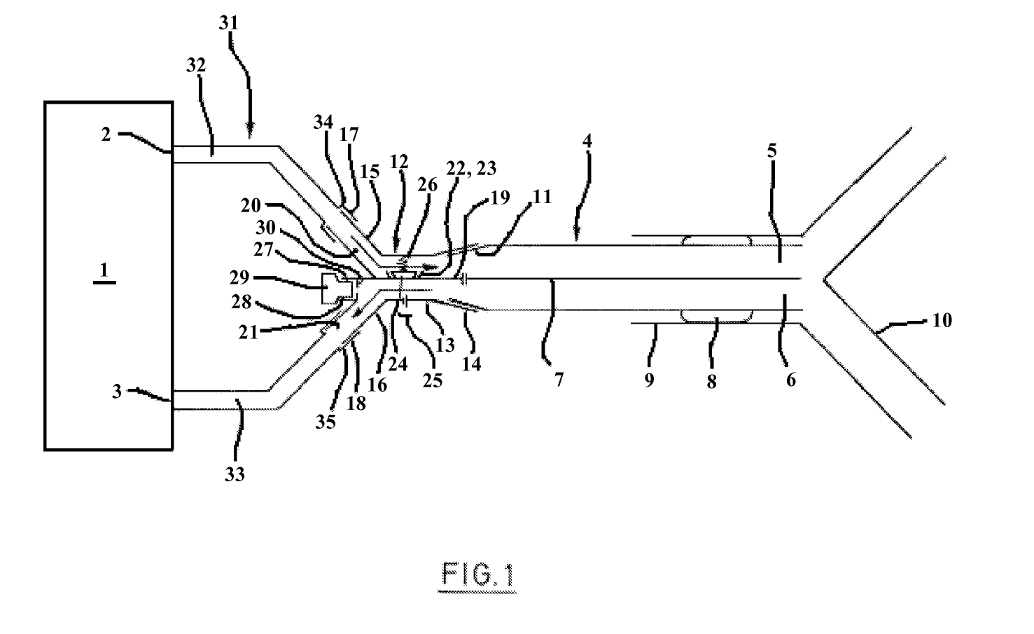

FIG. 1 a device for ventilation in a schematic view;

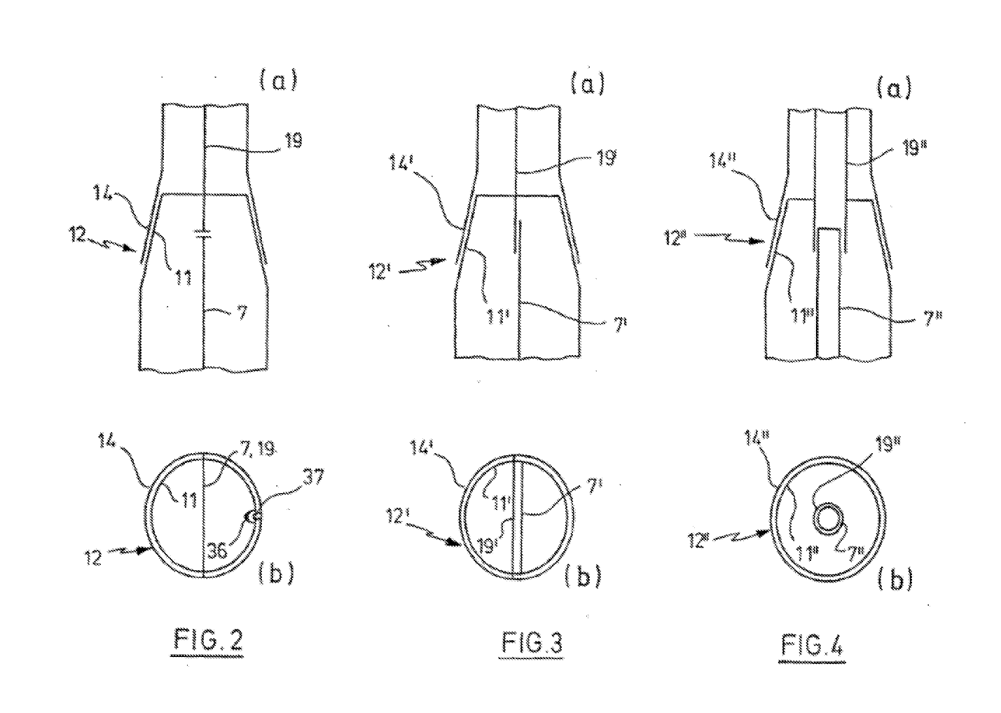

FIGS. 2a and b the connection between tubing system and ETT of said device in an enlarged partial longitudinal section (FIG. 2a) and in a cross section through the connecting region (FIG. 2b);

FIGS. 3a and b an alternative connection between tubing system and ETT in a partial longitudinal section (FIG. 3a) and in a cross section through the connecting region (FIG. 3b);

FIGS. 4a and b an alternative connection between tubing system and ETT in a partial longitudinal section (FIG. 4a) and in a cross section through the connecting region FIG. 4b);

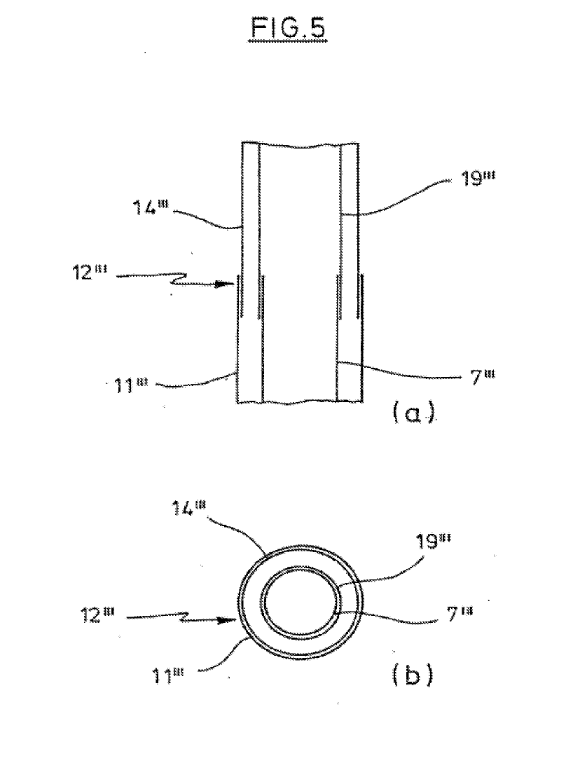

FIGS. 5a and b an alternative connection between tubing system and ETT in a partial longitudinal section (FIG. 5a) and in a cross section through the connecting region (FIG. 5b)

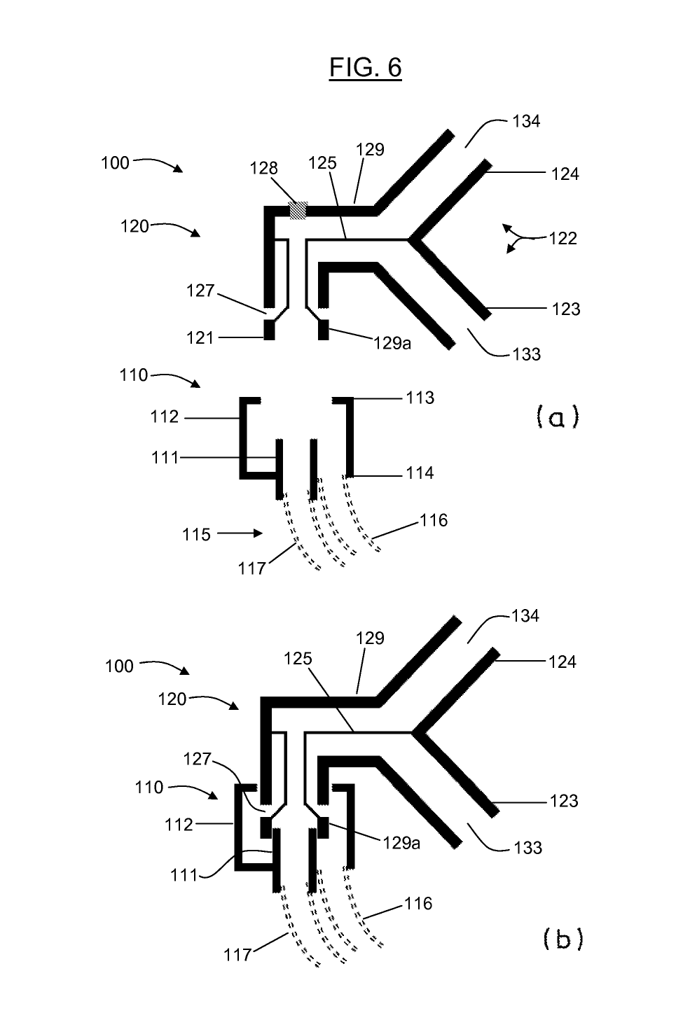

FIGS. 6a and b longitudinal sections of an embodiment of a two piece double lumen connector device that allows for cross connectivity, the two pieces being separated (FIG. 6a) and in an exemplary attachment (FIG. 6b)

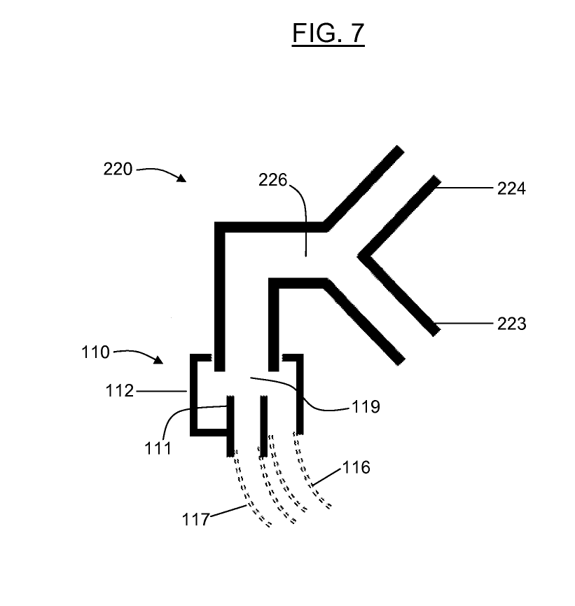

FIG. 7 a longitudinal section of an embodiment of a two piece double lumen connector wherein the connector piece is absent and the joining piece is attached to a standard, single lumen ventilator connector and shows illustrates cross-connectivity between a double lumen endotracheal tube and a standard ventilator connector;

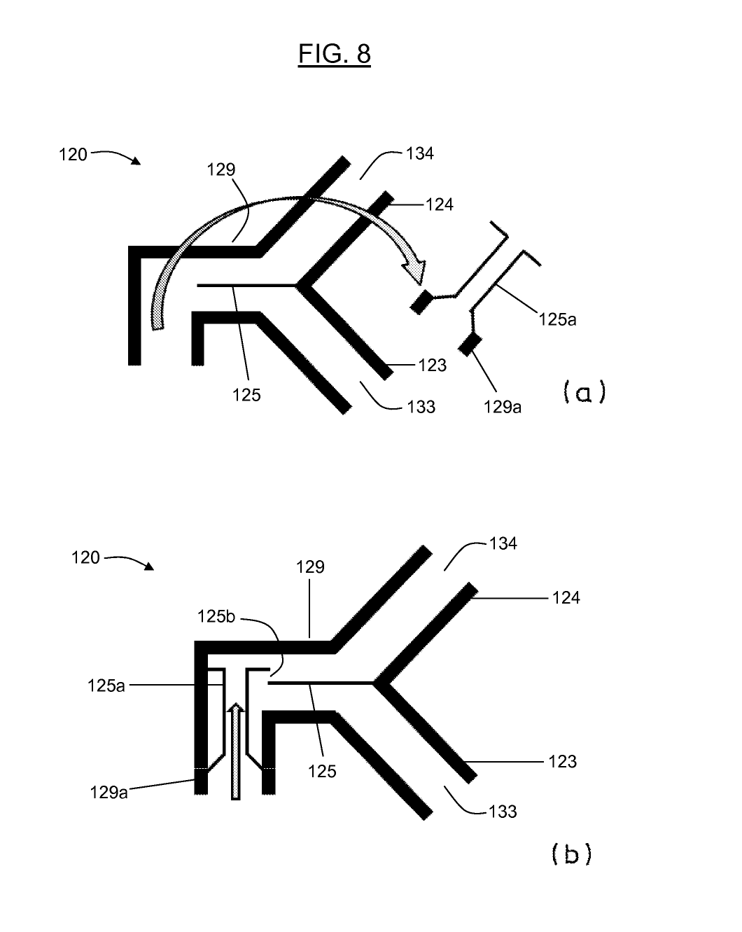

FIGS. 8a and b longitudinal sections of a connector piece from a two piece double lumen connector wherein the joining piece is absent and functionality of the connector piece in cross connectivity is illustrated by translocation of a section of the partition wall outside of the connector piece—i.e., removal of the section (FIG. 8a) and by translocation of the section of the partition wall within the connector piece—i.e., sliding of the section (FIG. 8b).

According to FIG. 1 the device for ventilation comprises of a ventilator 1 with an outlet 2, at which a stream of gas is provided for ventilation. Furthermore, the ventilator 1 has an inlet 3, to which a stream of exhaled gas can be conducted.

At the inlet 3 the ventilator 1 has an active valve, which is closed during inspiration in order to preserve the pressure in the lungs, and which is opened for expiration. Behind the valve the expired gas can be released into the surroundings. Alternatively the expired stream of gas can be reconditioned within the ventilator 1 (CO₂) and the gas needed for ventilation can be channeled back to the outlet 2, potentially adding fresh gas for ventilation.

Furthermore, there is an endotracheal tube 4 featuring in a tube-like body two lumina 5, 6, which are separated from each other by an axial wall 7. At its tracheal end this double lumen ETT 4 features an inflatable cuff 8 providing a seal toward the trachea 9 when the ETT 4 is advanced to shortly before the bronchi 10.

At the end distal to the patient the ETT 4 features a joining piece 11 in form of a cone. The partition wall 7 of the ETT is extended into the joining piece 11, ending at approximately half the height of the cone. Furthermore, there is a Y-shaped connector 12. It has in an essentially tubular connecting section 13 a conical opening 14, which is attached to the connecting piece 11 forming a seal.

The connecting section 13 features two separate tube nozzles 15, 16 with tubing connectors 17, 18 at their ends. Furthermore, the connector 12 has another partition wall 19, axially extending approximately from the middle of the conical opening 14 to the intersection of the two nozzles 15, 16. By this the connector 12 is separated into two chambers, one of which 20 extends from the inlet connector 17 into the opening 14, the other extending from the opening 14 towards the outlet connector 18.

Within the other partition wall 19 there is a shutter 22 with a valve seat 23 and a conical valve 24. It can be opened against the force of a spring 26 by a lever 25 extended to the outside.

The outlet chamber 21 features a tube 27 between the nozzles 15, 16, the axis of which is oriented towards the tubular connecting section 13, and which has an opening 28. The opening 28 is closed by a fastener 29. Beneath this fastener the tube 27 features an elastic perforated membrane.

The ventilator 1 is connected to the ETT 4 via a tubing system 31 and the connector 12. The tubing system 31 features an inspiratory tube 32 connecting the ventilator outlet 2 with the connector 17. Thereby the ventilator outlet 2 is connected to the inspiratory lumen 5 of the ETT 4. Furthermore, the tubing system 31 features an expiratory tube 33 connected to the connector 18. In the example the connections 34, of the tubes 32, 33 with the connectors 17, 18 can be freely rotated around the axes of the nozzles 15, 16.

FIG. 2 shows the partition wall and the other partition wall extending diagonally through the connecting piece and the opening 14. When the connecting piece 11 is introduced into the opening 14 forming a seal, the partitioning walls meet bluntly and form a seal, as well. A sealing connection between connecting piece 11 and opening 14 is only feasible in one rotational position, which is defined by an axial groove in the outside of the connecting piece 11 and an axial projection at the inside of the opening 14. Thereby the connections of chamber 20 with lumen 5 and chamber 21 with lumen 6 is guaranteed at all times.

In this way the ventilator 1 can be connected to the ETT 4 by merely sticking the single connector 12 onto the connecting piece 11. The connections of outlet 2 to the inspiratory lumen 5 and the inlet 3 to the expiratory lumen 6, which have different cross sections, is secured. If necessary a conventional Y-connector can be connected to the ETT 4, as well.

After removing the fastener 29 a suction catheter can be introduced into the expiratory lumen 6 down towards the bronchi 10, the perforated membrane 30 around it preserving the seal.

The embodiment in FIG. 3 differs from the one described above by the partition wall 7′ and the other partition wall 19′ being positioned slightly off the center and forming a seal in a side by side position when the opening 14 is stuck to the connecting piece 11. This arrangement at the same time makes sure that the connector 12′ and the connecting piece 11 can only be joined with a defined orientation.

The embodiment in FIG. 4 differs from the ones described above by the partition wall 7″ and the other partition wall 19″ each being constructed as axially oriented tubes. Preferably the tube 7″ is connected to the inspiratory lumen 5 and the tube 19″ to the connector 17. The surrounding ring-shaped spaces of the connecting piece 11 and the opening 14 are connected to the expiratory lumen 6 and the connector 18, respectively. Connectors 12″ and connecting pieces 11″ constructed in this way can be joined in arbitrary rotational orientation.

The embodiment in FIG. 5 differs from the ones described above by the cylindrical shape of the connecting piece 11′″ and opening 14″′ in their region of contact. Furthermore the opening 14″′ is inserted into the connecting piece 11″′. For this purpose the opening 14″′ has an external diameter of approximately 22 mm, the connecting piece 11′″ having a corresponding internal diameter.

Like in the embodiments described above the tube 7″′ is introduced into the tube 19″′, the two tubes 7″′ and 19″′ featuring a wider diameter of approximately 15 mm.

Preferably the interior lumen of the tubes 7″′ and 19′″ is used for expiration and the ring shaped exterior lumen between the tubes 7″′ and 19″′ on the one hand and connecting piece 11′″ and opening 14″′ on the other for inspiration.

The embodiments of FIG. 6, illustrate a double lumen connector device 100 having a two piece construction formed of a joining piece 110 and a connector piece 120. The joining piece 110 is formed of an inner tube 111 and an outer tube 112, the two tubes being axially oriented and spaced apart. The joining piece can be characterized as having a machine end 113 and a patient end 114, which is configured for attachment to a double lumen endotracheal tube. As illustrated, an expiratory lumen 117 from a double lumen endotracheal tube is connected to the patient end of the inner tube 111 so as to have a fluid communication through the space defined by the interior walls of the inner tube, and an inspiratory lumen 116 from the double lumen endotracheal tube is connected to the patient end of the outer tube 112 so as to have a fluid communication through the space defined by the interior walls of the outer tube and the exterior walls of the inner tube. Although the invention as described in relation to FIG. 6—FIG. 7 is described in terms of a joining piece configured as axially oriented and spaced apart inner and outer tubes, the invention should not be viewed as being limited to such construction. For example, the first and second tubes may be oriented side-by-side, as otherwise illustrated in the embodiment of FIGS. 2a and 2b. Other orientations of the first and second tubes of the joining piece also are encompassed to the extent that the two tubes otherwise provide the further characteristics otherwise described herein for facilitating standardized connections and cross-connectivity between single lumen devices and double lumen devices.

The connector piece 120 includes an exterior wall 129 that defines the piece, which includes a patient end 121 and a machine end 122. The machine end of the connector piece comprises an inspiratory tube nozzle 123 and an expiratory tube nozzle 124 in a Y-shaped configuration (although other configurations—e.g., T-shaped—are encompassed) such that the nozzles intersect so as to form a first chamber 133 and a second chamber 134. In the illustrated embodiment, the first chamber 133 can be characterized as an inspiratory chamber (extending within the inspiratory tube nozzle 123), and the second chamber 134 can be characterized as an expiratory chamber (extending within the expiratory tube nozzle 124). The two chambers are separated by a partition wall 125 that extends from within the connector piece to the patient end 121 of the connector piece.

The joining piece 110 is configured such that the outer tube 112 extends a distance beyond the inner tube 111 at the machine end 113 of the joining piece. As shown in FIG. 6b particularly, this construction with a longer outer tube allows the outer tube to slide around the exterior wall 129 of the patient end 121 of the connector piece 120. During such engagement, the exterior wall of 129 of the connector piece 120 also interacts with the inner tube 111 of the joining piece 110 such that the inner wall of the joining piece slides within the patient end 121 of the connector piece. As so joined, two isolated flow paths are formed. Expiratory air passes from the expiratory lumen 117 of the endotracheal tube, through the space within the inner tube 111 of the joining piece 110, into the second chamber defined by the partition wall 125, and out of the expiratory tube nozzle 124.

Conversely, inspiratory air flows into the inspiratory tube nozzle 123, through the first chamber 133 defined by the partition wall 125 and the exterior wall 129 of the connector piece 120, through the space defined by the interior wall of the outer tube 112 and the exterior wall of the inner tube 111, and into the inspiratory lumen 116 of the endotracheal tube. In other embodiments, the flow paths may be reversed such nozzle 123 can be the expiratory nozzle and the pathway encompassing nozzle 123 and the first chamber 133 can be the expiratory pathway. Likewise, nozzle 124 can be the inspiratory nozzle and the pathway encompassing nozzle 124 and the second chamber 134 can be the inspiratory pathway. The former configuration can be preferred, particularly in embodiments such as shown in FIG. 6a wherein the exterior wall of the connector piece 120 includes a removable fastener 128 that when removed, provides a passage that can allow introduction of a catheter or other suction means or other desired instruments. In the formed configuration, the illustrated position of the fastener 128 can provide for direct access through the expiratory pathway into the bronchi. A membrane or other means for maintaining a seal within the device may be included, as already described herein. Such closeable opening in the connector piece, when opened, can thus allow access into the inner tube of the joining piece, or, if desired the outer tube of the joining piece.

The connection between the joining piece 110 and the connector piece 120 can be facilitated by configuring the pieces to have standardized sizing of components. Specifically, as illustrated in this embodiment, the opening at the machine end 113 of the joining piece 110 has an internal diameter of about 22 mm, and the connector piece 120 at the patient end has an external diameter of about 22 mm such that the pieces conveniently slide together and may likewise interact with other devices having the same, standardized sizings. Similarly, the inner tube 111 of the joining piece 110 has an external diameter of about 15 mm, and the connector piece 120 at the patient end has an internal diameter of about 15 mm such that inner tube conveniently slides within the connector piece. Sizing of the openings on the patient side of the joining piece may be chosen so as to facilitate connection with lumina of any size provided with an endotracheal tube (e.g., different sized endotracheal tubes that may be provided for men, women, and children).

The difference in the lengths of the inner tube and the outer tube at the machine end of the joining piece provides for multiple advantages. In one embodiment, the exterior wall 129 of the connector piece 120 includes one or more apertures 127 near the patient end 121 thereof. Such aperture provides fluid communication into the first chamber 133. Thus, as shown in FIG. 6b, the sealing engagement of the exterior of the inner tube 111 with the interior of the connecting piece 120 forms an isolated flow path, as described previously, through the inner tube and the second chamber. Thus, the second chamber of the connector piece and the inner tube of the joining piece may be configured to be sealing engaged and form an expiratory limb. Such engagement, in combination with the sealing engagement of the exterior of the connector piece 120 and the inner surface of the opening at the machine end 113 of the outer tube 112, forms an isolated flow path, as described previously, through the space defined by the exterior of the inner tube and the interior of the outer tube, through the apertures 127 in the connector piece 120, and through the first chamber 133. Thus, the first chamber of the connector piece and the outer tube of the joining piece may be configured to be sealing engaged and form an inspiratory limb. The distance between the apertures and the terminal end of the connector piece at the patient end can vary depending upon the distance between the terminal ends of the inner tube and the outer tube at the machine end of the joining piece. When fully connected, the outer wall of the joining piece preferably extends up the connector piece past the apertures. In one embodiment, the position of the apertures on the connector piece and the distance between the terminal ends of the inner and outer tubes of the joining piece can be such that, when fully connected such that the terminal end of the inner tube connects with the terminal end of the connector piece and the terminal end of the outer tube slides up the connector piece beyond the terminal end thereof, the apertures of the connector piece are approximately midway between the terminal end of the inner tube and the terminal end of the outer tube (e.g., about 30% to about 70% of the distance between the terminal ends of the inner and outer tubes, as measured from either terminal end).

As seen in FIG. 7, the joining piece 110 can be removed from the connector piece and can be used to provide cross connectivity with a standard, single lumen ventilator connector 220. Such standard ventilator connector includes an inspiratory nozzle 223 and an expiratory nozzle 224 that meet to form a single, common chamber 226 within the connector. The double lumen connector device is advantageous in that, if necessary, it may be used to connect a double lumen endotracheal tube with a standard ventilator connector as shown. In such embodiment, the expiratory lumen 117 from the double lumen endotracheal tube is again connected to the patient end of the inner tube 111 so as to have a fluid communication through the space defined by the interior walls of the inner tube, and the inspiratory lumen 116 from the double lumen endotracheal tube is connected to the patient end of the outer tube 112 so as to have a fluid communication through the space defined by the interior walls of the outer tube and the exterior walls of the inner tube. As will be appreciated, such connection of the lumens to the joining piece may be reversed. The double lumen endotracheal tube thus may be connected to a standard ventilator connector because the machine end of the outer tube 112 has an interior diameter that corresponds to the standard exterior diameter at the patient end of typical, standard ventilator connectors. Unlike the connection with a double lumen connector piece, the joining piece 110 only slides past the terminal end of the standard ventilator connector 220 a distance that is less than the distance between the terminal end of the inner tube and the terminal end of the second tube. Thus, as seen, a mixing head space 119 is provided wherein expiratory air from the expiratory lumen 117 has open access to the common chamber 226 of the standard ventilator connector. Likewise, the mixing head space 119 provides open access for the inspiratory air from the common chamber 226 into the inspiratory lumen 116. Accordingly, in some embodiments, a double lumen endotracheal tube that cross connects with a patent end of a standard, single lumen ventilator connection piece can be provided as a combination of a double lumen endotracheal tube and the joining piece 110. Such endotracheal tube thus may cross connect with the single lumen connector, as now described, or be combined with the double lumen connector piece 120 for double lumen ventilation.

The connector piece 120 may similarly be used apart from the joining piece to allow for cross connectivity with standard, single lumen patient breathing components—e.g., a single lumen endotracheal tube or a ventilation mask. Again, the standard sizing at the patient end of the connector piece (i.e., 22 mm exterior diameter and 15 mm interior diameter) facilitates such cross connectivity. There still remains the need, however, to not allow the partition wall 125 to impede air flow in cross connectivity.

In FIG. 8, two embodiments are illustrated that handle this aspect of the device. Specifically, the partition wall 125 can be configured with a translocatable section 125a. In FIG. 8a, the translocatable section 125a is configured for translocation from a first position (see FIG. 6a) wherein the first chamber 133 is separated from the second chamber 134 to a second position that is outside of the connector piece. In other words, the translocatable section 125a is removable. In the illustrated embodiment, the translocatable section 125a remains connected to a segment 129a of the exterior wall 129 of the connector piece 120. It can be preferably for the translocatable section of the partition wall to be in sufficient proximity to the patient end of the connector piece to allow manual translocation via access through the patient end of the connector. This may include removing translocatable section 125a and the exterior wall segment 129a together. In FIG. 8b, the translocatable section 125a is configured for translocation from a first position (see FIG. 6a) wherein the first chamber 133 is separated from the second chamber 134 to a second position that is still inside the connector piece but that also allows fluid communication between the chambers within the connector piece. As seen in FIG. 8b, the translocatable section slides further into the connector piece so that a separation 125b is made in the partition wall, such separation allowing fluid connection between the first and second chambers. Other means of translocation also are encompassed. In the illustrated embodiment, the exterior wall segment 129a likewise slides and can partially or completely eliminate the aperture 127.

1. A double lumen connector device providing cross-connectivity between a ventilation device and a single lumen or double lumen patient side breathing component, the double lumen connector device comprising:
a joining piece comprising: first and second tubes; a machine end; and a patient end that is configured for attachment to a double lumen endotracheal tube; wherein the first tube has a first standardized external diameter; the second tube has a first standardized internal diameter; and the second tube at the machine end extends beyond the first tube at the machine end; and
a connector piece comprising: a patient end that slidably engages the machine end of the joining piece; a machine end having separate inspiratory and expiratory tube nozzles extending therefrom and intersecting within the connector piece; and first and second chambers formed within the connector piece by the intersecting nozzles, the chambers remaining separated from each other by a partition wall that extends from within the connector piece to the patient end of the connector piece; wherein the patient end of the connector piece has a second standardized internal diameter corresponding to the first standardized external diameter and has a second standardized external diameter corresponding to the first standardized internal diameter.

2. The device of claim 1, wherein the first and second tubes are configured to be axially oriented and spaced apart as inner and outer tubes, respectively, and wherein the outer tube at the machine end extends beyond the inner tube at the machine end.

3. The device of claim 1, wherein the first standardized external diameter and the second standardized internal diameter are each about 15 mm.

4. The device of claim 1, wherein the first standardized internal diameter and the second standardized external diameter are each about 22 mm.

5. The device of claim 1, wherein the second chamber of the connector piece and the first tube of the joining piece are configured to be sealingly engaged.

6. The device of claim 5, wherein the engaged second chamber and first tube define an expiratory limb.

7. The device of claim 1, wherein the first chamber of the connector piece and the second tube of the joining piece are configured to be sealingly engaged.

8. The device of claim 7, wherein the engaged first chamber and second tube define an inspiratory limb.

9. The device of claim 1, wherein the connector piece further comprises a closable opening which, when opened, allows access into the first tube of the joining piece.

10. The device of claim 1, wherein a section of the partition wall within the connector piece is configured for translocation from a first position wherein the chambers remain separated within the connector piece to a second position allowing fluid communication between the chambers within the connector piece.

11. The device of claim 10, wherein the second position is within the connector piece.

12. The device of claim 10, wherein the second position is outside of the connector piece.

13. The device of claim 10, wherein the section of the partition wall that is configured for translocation is in sufficient proximity to the patient end of the connector piece to allow manual translocation via access through the patient end of the connector.

14. The device of claim 1, wherein the ventilation device is selected from the group consisting of a ventilator, an anesthesia machine, and a resuscitation bag.

15. The device of claim 1, wherein the patient side breathing component is a single lumen endotracheal tube.

16. The device of claim 1, wherein the patient side breathing component is a breathing mask.

17. A double lumen endotracheal device that cross-connects with a patient end of a standard, single lumen ventilator connection piece comprising:
a double lumen endotracheal tube comprising a first lumen and a separate, second lumen, the double lumen endotracheal tube having an end that is proximal to a patient and an end that is distal to the patient; and
a joining piece comprising: axially oriented and spaced apart inner and outer tubes;
a machine end that is configured for attachment to the standard, single lumen ventilator;
and a patient end that is configured for attachment to the end of the double lumen endotracheal tube that is distal to the patient; wherein the inner tube has a first standardized external diameter; the outer tube has a first standardized internal diameter that corresponds to an external diameter of the patient end of the standard, single lumen ventilator connection piece; and the outer tube at the machine end extends beyond the inner tube at the machine end a sufficient distance such that, when the joining piece is connected to the patient end of the standard, single lumen ventilator connection piece, the inner tube of the joining piece remains spaced apart therefrom.

18. The double lumen endotracheal tube of claim 17, wherein the first standardized external diameter is about 15 mm.

19. The double lumen endotracheal tube of claim 17, wherein the first standardized internal diameter is about 22 mm.

20. The double lumen endotracheal device of claim 17, wherein the joining piece is separable from the double lumen endotracheal tube.

21. The double lumen endotracheal device of claim 17, wherein the joining piece is integrally formed with the double lumen endotracheal tube.

22. A ventilator connector piece configured for cross connectivity of a ventilation device with a single lumen endotracheal tube or a double lumen endotracheal tube, the connector piece comprising: a patient end; a machine end having separate inspiratory and expiratory tube nozzles extending therefrom and intersecting within the connector piece; and first and second chambers formed within the connector piece by the intersecting nozzles, the chambers remaining separated from each other by a partition wall that extends from within the connector piece to the patient end of the connector piece; wherein a section of the partition wall within the connector piece is configured for translocation from a first position wherein the chambers remain separated within the connector piece to a second position allowing fluid communication between the chambers within the connector piece.

23. The connector device of claim 22, wherein the second position is within the connector piece.

24. The connector device of claim 22, wherein the second position is outside of the connector piece.

25. The connector device of claim 22, wherein the section of the partition wall that is configured for translocation is in sufficient proximity to the patient end of the connector piece to allow manual translocation via access through the patient end of the connector.

26. The connector device of claim 22, wherein the patient end has in internal diameter of about 15 mm and an external diameter of about 22 mm.
